(19) United States
(12) Patent Application Publication
(10) Pub. No.: US 2006/0029961 A1
(43) Pub. Date: Feb. 9, 2006

(54) Patterned surfaces and their use in diffraction-based sensing

(76) Inventors: M. Cynthia Goh, Toronto (CA); Jane B. Goh, Toronto (CA); Richard Loo, Toronto (CA)

Correspondence Address: Ralph A. Dowell of DOWELL & DOWELL P.C., 2111 Eisenhower Ave, Suite 406, Alexandria, VA, 22314, US

(21) Appl. No.: 11/196,483

(22) Filed: Aug. 4, 2005

Related U.S. Application Data
(60) Provisional application No. 60/598,438, filed on Aug. 4, 2004.

(51) Int. Cl. C12Q 1/68; G01N 33/53; C12M 1/34

(52) U.S. Cl. 435/6; 435/7.1; 435/287.2

(57) ABSTRACT

Fabrication of surfaces patterned with chemical crosslinkers for surfaces patterned with chemical crosslinkers for solution-phase immobilization of probe molecules and their use in diffraction-based sensing. In one embodiment of the invention, a chemical crosslinker, X¹—R¹—Y¹, is deposited on areas of the substrate surface that defines a pattern and allowed to react with the surface for a sufficient period of time to attain the desired density of covalently linked crosslinkers on the surface. The reaction between the crosslinker X¹—R¹—Y¹ and the surface can be accelerated using known techniques such as heating, microwave irradiation, sonication, etc, to achieve the desired density in less time. In another embodiment of the invention, two or more other types of cross-linkers may also be laid down in patterns on the surface to detect for two or more other types of molecules in solution.

### CROSS REFERENCE TO RELATED U.S. PATENT APPLICATIONS

This patent application relates to U.S. provisional patent application Ser. No. 60/598,438 filed on Aug. 4, 2004 entitled PATTERNED SURFACES AND THEIR USE IN DIFFRACTION-BASED SENSING, which is incorporated herein by reference in its entirety.

### FIELD OF THE INVENTION

The present invention relates to fabrication of surfaces patterned with chemical crosslinkers for solution-phase immobilization of probe molecules and their use in diffraction-based sensing.

### BACKGROUND OF THE INVENTION

Diffraction-based sensors rely on being able to fabricate a substrate surface patterned with probe molecules that are biologically active. Patterning of surfaces can be accomplished in many ways. Among the many different methods, one of the most practical is microcontact printing. This method involves using an elastomeric stamp having a surface relief pattern, inking the stamp with a solution of molecules, and putting the stamp in contact with the surface of the substrate to be patterned, thereby transferring the molecules in areas of contact between the stamp and the substrate surface. U.S. Pat. No. 5,512,131 to Kumar et. al. describes the formation of patterned surfaces by microcontact printing of molecules that form self-assembled monolayers (SAM) on surfaces, with gold as the sole example of surface used. U.S. Pat. No. 6,444,254 to Chilkoti and Yang describes the patterning by microcontact printing of ligands on activated polymer surfaces, said ligands containing a reactive end that binds covalently to the surface of the activated polymer. The ligands are described as either biological molecules or non-biological synthetic polymers and plastics. The direct microcontact printing of proteins onto silicon, silicon dioxide, polystyrene, glass and silanized glass is reported in Bernard, A; Delamarche, E.; Schmid, H.; Michel, B.; Bosshard, H. R.; Biebuyck, H.; “Printing Patterns Of Proteins” Langmuir (1998) 14, 2225-2229.

U.S. Pat. No. 5,922,550 (Biosensing devices which produce diffraction images) describes a method of producing a patterned surface by microcontact printing of a self-assembled monolayer of receptors on a metal-coated polymer. This is extended to the case of a predetermined pattern of receptors (not necessarily self-assembling) in U.S. Pat. No. 6,060,256 (Optical Diffraction Biosensor).

All these patents describe the direct patterning of probe molecules on surfaces by microcontact printing. While microcontact printing appears to work well for patterning of small molecules, for example alkanethiols and ligands, proteins tend to be rendered biologically inactive during the process.

The use of heterobifunctional chemical crosslinkers for the conjugation of proteins and other biomolecules to other proteins, small molecules, polymers, fluorescent tags, etc is widely known and does not result in the loss of biological activity (See Bioconjugate Techniques, G T Hermanson, Academic Press 1996). Hence, patterning of these chemical crosslinkers on surfaces and the subsequent solution-phase covalent reaction of proteins and other probe molecules with these crosslinkers should result in immobilized biomolecules with high biological activity.

The use of patterned surfaces in diffraction-based assays has been described. U.S. Pat. No. 5,922,550 (Biosensing devices which produce diffraction images) describes a device and method for detecting and quantifying analytes in a medium based on having a predetermined pattern of self-assembling monolayer with receptors on a polymer film coated with metal. The size of the analytes is of the same order as the wavelength of transmitted light, thereby its binding results in a diffraction pattern that is visible. U.S. Pat. No. 4,647,544 (Immunoassay using optical interference detection) describes a light optical apparatus and method, in which a ligand, or an antibody, is arranged in a predetermined pattern, preferably stripes, on a substrate, and the binding between the ligand and an antiligand, or between the antibody and an antigen, is detected by an optical detector set at the Bragg scattering angle, which is expected to arise due to optical interference. The pattern of ligand or antibody is created by first laying out a uniform layer of antibody on a substrate, then deactivating sections of this coverage. U.S. Pat. No. 4,876,208 (Diffraction immunoassay apparatus and method) describes the apparatus and reagents for an immunoassay based on a silicon or polysilicon substrate with a pattern of evenly spaced lines of a biological probe (a ‘biological diffraction grating’) to which binding can take place. The pattern is created by first coating the substrate with an even layer of antibodies, then deactivating regions by the use of a mask and of ultraviolet (UV) lights. This idea is extended to the assay of DNA in U.S. Pat. No. 5,089,387 (DNA probe diffraction assay and reagents), which describes a biological diffraction grating, and a process for its manufacture by first immobilizing a uniform layer of hybridizing agent on a smooth surface, and then exposing this surface to UV radiation through a mask with diffraction grating lines. The UV exposure deactivates the hybridizing agent, leaving a pattern of lines of active hybridizing agents.

U.S. Pat. No. 5,512,131 to Kumar et. al. describes the use of a surface patterned with a SAM as a biosensor whereby the SAM provided with a binding partner of an analyte can be exposed to a medium containing the analyte mixed with a known quantity of labeled analyte (competitive assay) or to a medium containing the analyte and an excess of a labeled secondary binding partner (sandwich assay) then “illuminated with coherent electromagnetic radiation and a diffraction observe, the intensity of the diffraction pattern being used to quantitate the amount of label.” The patent describes the detection of a labeled analyte that has been synthetically incorporated into the medium and failed to provide means of detecting the real analyte.

The present invention addresses the issue of patterning of probe molecules, such as proteins, on surfaces by fabrication of a substrate with a surface containing patterned chemical crosslinkers. The patterning of the probe molecules is done in solution thus ensuring the retention of their biological activity. Also addressed is the use of these patterned surfaces as sensors in diffraction-based assays.

### SUMMARY OF THE INVENTION

The present invention provides a sensor for immobilizing at least one type of probe molecules in patterns on a substrate, comprising:

a substrate having a surface with pre-selected areas of the surface patterned with at least one chemical crosslinker, X¹—R¹—Y¹, wherein X¹ is a chemical functional group that can chemically bind with the surface, R¹ is a chemical moiety that serves as a spacer to provide distance between the surface and the probe molecules to be immobilized and also reduce non-specific interactions, and Y¹ is a chemical functional group which can form a strong interaction, either covalent or non-covalent, with the probe molecules;

remaining areas of the substrate not patterned with the at least one chemical crosslinker X¹—R¹—Y¹ being coated with blocking molecules, X²—R², wherein X² is a chemical functional group that can covalently react with the surface which may or may not be the same as X¹, and R² is a chemical moiety that reduces non-specific interactions and may or may not be the same as R¹, wherein contacting the patterned surface with the probe molecules in solution effects immobilization of the probe molecules through a strong interaction between the probe molecules and the Y¹-chemical functional group of the at least one chemical crosslinker X¹—R¹—Y¹.

The present invention also provides a method for fabricating substrates with immobilized probe molecules in a pattern, comprising:

patterning pre-selected portions of a surface of a substrate with chemical crosslinkers, X¹—R¹—Y¹, wherein X¹ is a chemical functional group that can covalently react with the surface, R¹ is a chemical moiety that serves as a spacer to provide distance between the surface and the probe molecules to be immobilized and also helps to minimize non-specific interactions, and Y¹ is a chemical functional group which can form a strong chemical interaction, either covalent or non-covalent, with the probe molecules;

exposing the substrate to blocking molecules, X²—R², wherein X² is a chemical functional group that can covalently react with the surface which may or may not be the same as X¹, and R² is a chemical moiety that helps minimize non-specific interactions and may or may not be the same as R¹ so that areas of the substrate not patterned with the crosslinker X¹—R¹—Y¹ is coated with the blocking molecules X²—R²; and

contacting the patterned surface with the probe molecules in solution to effect strong chemical interaction between the Y¹ chemical functional groups of the cross linkers and the probe molecules thereby immobilizing the probe molecules attached thereto.

In another aspect of the present invention there is provided a method for fabricating a substrate with immobilized probe molecules in a pattern, comprising:

patterning pre-selected portions of a surface of the substrate with at least two types of chemical crosslinkers, X¹—R¹—Y¹ and X³—R³—Y³, wherein patterns defined by the two crosslinkers are different and distinct from each other, wherein X¹ and X³ are chemical functional groups that can covalently react with the surface and may or may not be the same, wherein R¹ and R³ *are chemical moieties that serve as spacers to provide distance between the surface and the probe molecules to be immobilized and also helps to minimize non-specific interactions and may or may not be the same, and wherein Y¹ and Y³ are chemical functional groups that can form strong interactions, either covalent or non-covalent, with the probe molecules and may or may not be the same;

remaining areas of the substrate not patterned with the chemical crosslinkers X¹—R¹—Y¹ being coated with blocking molecules, X²—R², wherein X² is a chemical functional group that can covalently react with the surface which may or may not be the same as X¹ or X³, and R² is a chemical moiety that helps minimize non-specific interactions and may or may not be the same as R¹ or R³, wherein contacting the patterned surface with first probe molecules in solution effects immobilization of the first probe molecules through a strong interaction between the first probe molecules and the y¹-functional group of the chemical crosslinkers, X¹—R¹—Y¹, and wherein contacting the patterned surface with a solution containing a second probe molecule effects immobilization of said second probe molecules through a strong interaction between the second probe molecules and the Y³-functional group of the chemical crosslinker X³—R³—Y³.

### BRIEF DESCRIPTION OF THE DRAWINGS

The invention will now be described, by way of example only, reference being had to the accompanying drawings, in which;

FIG. 1 is a top view of a substrate having a pattern of chemical crosslinker, X¹—R¹—Y¹ laid out in a unique pattern on the surface with the remainder of the surface being passivated with a blocking agent X²—R²; and

FIG. 2 is a top view of a substrate having two patterns of chemical crosslinkers, X¹—R¹—Y¹ and X³—R³—Y³, each laid out in a unique pattern on the surface with the remainder of the surface being passivated with a blocking agent X²—R².

### DETAILED DESCRIPTION OF THE INVENTION

The following terminology will be used in accordance with the given definitions to describe the invention:

A probe molecule is a molecule that is capable of binding selectively to another molecule, examples of which are antibodies, antigens, oligonucleotides, etc.

An alkyl chain is a straight or branched chain of saturated carbon atoms. A cycloalkyl group is a cyclic structure of saturated carbon atoms. An aryl group is an aromatic moiety containing 5 to 6 atoms of carbon and/or heteroatoms such as nitrogen, oxygen or sulfur per ring, and may be composed of one or more rings that are fused or linked. A halo group is used to refer to either chloro, bromo, fluoro, or iodo moiety.

A protecting group is a chemical moiety that is used to temporarily inactivate a functional group to prevent its interference with another reaction. Orthogonal protecting groups are protecting groups that can be deprotected individually without affecting the others.

A substrate surface is any exterior area of a monolithic material, be it the material itself or a coating upon the material. The substrate surface can be glass, polymer, or metal. The coating can be introduced using a variety of ways, including chemical and physical deposition in the vapor phase or in solution.

Polymer surfaces can be polystyrene, styrene-maleic anhydride copolymer, styrene-acrylonitrile copolymer (SAN), polycarbonate, polyethylene terephthalate (PET), polylactic acid, polyglycolic acid, polyvinyl alcohol, polyglutamic acid, polylysine, and polyethylene glycol.

Regardless of the composition of the monolith material, the substrate surface will contain functional groups, including nucleophiles, electrophiles, free-radical-producing, alkenyl, alkynyl, photo-activated, that can readily react with the chemical functional group X on the chemical crosslinker, or can be activated in situ prior to reaction with the chemical crosslinker. Examples of nucleophilic functional groups on the substrate surface are amines, hydroxyls, hydrazides, and thiols. Examples of electrophilic functional groups are carboxylic acids and all their activated forms including, but not limited to, anhydrides, acid chlorides, N-hydroxy succinimide, and imidazolide, alpha-halo carbonyls, epoxides, aldehydes, isocyanate, and isothiocyanate.

In one embodiment of the invention, a chemical crosslinker, X¹—R¹—Y¹, is deposited on areas of the substrate surface that defines a pattern and allowed to react with the surface for a sufficient period of time to attain the desired density of covalently linked crosslinkers on the surface. The reaction between the crosslinker X¹—R¹—Y¹ and the surface can be accelerated using known techniques such as heating, microwave irradiation, sonication, etc, to achieve the desired density in less time.

X¹ is a chemical functional group that can covalently react with the substrate surface. For electrophilic surfaces, X¹ will be nucleophilic and may include amines, hydrazides, hydroxylamines, or thiols. For nucleophilic surfaces, X¹ will be electrophilic, and includes carboxylic acids and all their activated forms, epoxides, trialkoxysilanes, dialkoxysilanes, and chlorosilanes. X¹ can also be light activated and/or free-radical-forming such as peroxides, azo, and azido.

R¹ is a moiety that is compatible with biomolecules and minimizes non-specific interactions. R¹ may preferably be composed of an alkyl chain, from 2 to about 200 atoms in length, which may or may not be interrupted by heteroatoms and/or aryl groups and/or cycloalkyl groups.

Y¹ is a chemical functional group that is responsible for immobilization of the probe molecules in solution, and can form a strong interaction, covalent or non covalent, with the probe molecule. In a preferred embodiment, Y¹ forms a covalent interaction with the probe molecules under conditions that do not severely affect the biological activity of the probe molecules.

In one embodiment, Y¹ is activated in situ. The activation procedure is dependent on the nature of Y¹ and would be obvious to those skilled in the art. In a preferred embodiment, Y¹ is a highly reactive functional group and does not require activation prior to reaction with the probe molecules. Included in this are epoxide, aldehyde, alpha-halo carbonyl, amine, hydrazide, isocyanate, and activated carboxylic acids, such as acid chloride, mixed anhydride, N-hydroxysuccinimidyl (NHS) ester, pentafluorophenyl (PFP) ester, hydroxybenzotriazole (HObt) ester, and imidazolide.

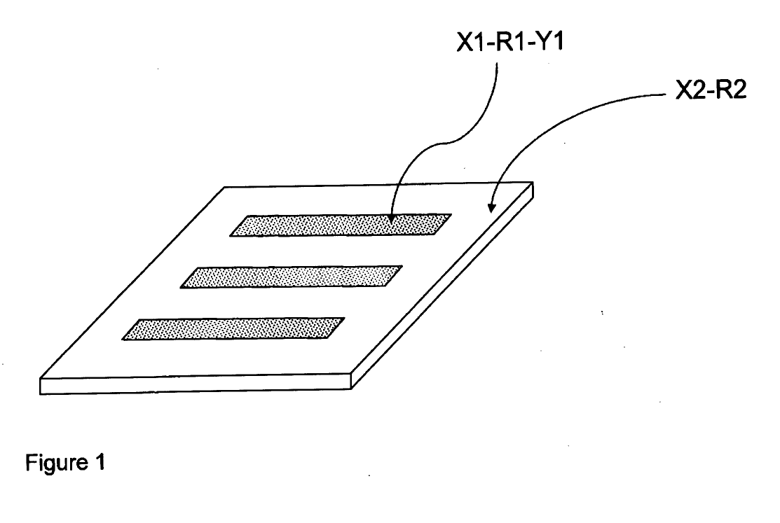

Referring to FIG. 1, in one embodiment of invention where the sensor is to be used to detect a single analyte, the remainder of the substrate surface not patterned with X¹—R¹—Y¹ is passivated with a blocking agent X²—R² where X² is a functional group capable of forming a covalent interaction with the substrate surface, and may or may not be the same as X¹. R² is a moiety that is compatible with biomolecules and minimizes non-specific interactions. R² may be composed of an alkyl chain, 2 to 200 atoms in length, which may or may not be interrupted by heteroatoms and/or aryl groups and/or cycloalkyl groups, and may or may not be the same as R¹.

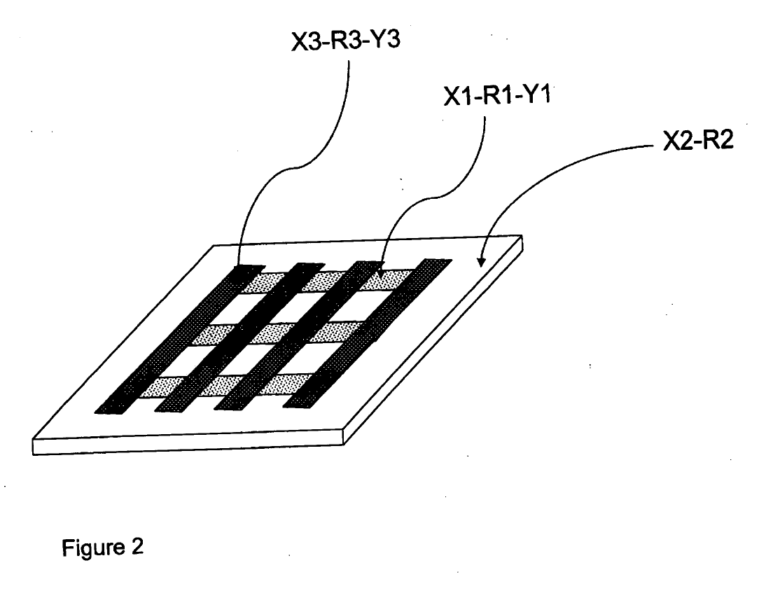

In another embodiment where the sensor is to be used for detection of at least two analytes, the patterning step is iterated such that at least two sets of crosslinkers are patterned on the same surface area of the substrate. Thus after patterning of X¹—R¹—Y¹ another crosslinker X³—R³—Y³ is deposited on areas of the substrate surface that defines a pattern different from that defined by X¹—R¹—Y¹ and allowed to react with the surface for a sufficient period of time to attain the desired density of covalently linked crosslinkers on the surface, see FIG. 2. The reaction between the crosslinker X³—R³—Y³ and the surface can be accelerated using known techniques such as heating, microwave irradiation, sonication, etc, to achieve the desired density in less time. X³ is a chemical functional group that may be chosen from the functional groups defined for X¹ and may or may not be the same as X¹. R³ may be chosen from the moieties defined for R¹ and may or may not be the same as R¹. Y³ is a chemical functional group that may be chosen from the functional groups defined for Y¹ and may be the protected or masked version of any of these functional groups. The protecting group is chosen so as to enable its deprotection under conditions that will not aversely affect the biological activity of the first set of probe molecules.

The step of patterning of crosslinkers may be iterated to produce a substrate surface patterned with multiple sets of crosslinkers. In practice, however, there is a finite number of iterations that can be done on one given area of the surface due to the limited number of different orthogonal protecting groups that can be used under the conditions necessary to preserve the biological activity of the other probe molecules already immobilized on the surface. In a particularly preferred embodiment, only two sets of crosslinkers are patterned on one given area.

After the substrate surface has been patterned with crosslinkers, it is passivated with the blocking agent as described above. After passivation, the patterned substrate surface is ready for use in solution-phase immobilization of probe molecules. In one embodiment, the patterned surface is contacted with the solution of probe molecules for a period of time sufficient to effect the reaction of the probe molecules with the crosslinkers. In another embodiment where the crosslinkers are activated in situ, the patterned surface is first contacted with a solution of the activating agent for a sufficient period of time, rinsed free of excess activating agent under conditions that do not deactivate the crosslinkers, then contacted with a solution of the probe molecules.

In one embodiment, the probe molecules may interact with the Y functional group of the crosslinker through any of the functional groups that are already on the probe molecules provided that the interaction does not result in loss of biological activity of the probe molecules. For example, in the case of proteins as probe molecules, these functional groups may be reactive amino acid residues comprising the protein, including the termini. The interaction between the probe molecules and the Y functional group of the crosslinkers may or may not be covalent, but is sufficiently strong to prevent washing off of the probe molecules during the assay. In a preferred embodiment, the interaction is covalent.

In another embodiment, the protein could interact through affinity tags that are introduced into the probe molecules through synthetic means. These affinity tags may be amino acid sequences such as polyhistidines, chemical crosslinkers, and other proteins, such as glutathione S-transferase, or streptavidin.

The interaction between the probe molecules and the functional groups on the surface may be such that another reagent can be added during the reaction to further enhance the interaction as in the case of the reaction between aldehydes and amines to give imines or Schiff bases. Addition of a reducing agent such as sodium cyanoborohydride in this case gives an amine linkage, which is more stable than the original Schiff base.

After the first set of probe molecules is immobilized, the remainder of the first set of crosslinkers that did not react with probe molecules may have to be blocked. This could be accomplished by contacting the substrate surface with a solution of the blocking agent X²R² or other blocking solutions known to those skilled in the art such as milk, solutions of albumin, salmon sperm, or herring sperm. For a substrate patterned with only one set of crosslinkers, the sensor is now ready for use in diffraction-based assay.

For immobilization of a second set of probe molecules, the Y functional groups of the second set of crosslinkers will have to be de-protected or unmasked. The conditions for de-protection or unmasking depends on the nature of the protecting groups and is known to those skilled in the art. After de-protection, the Y functional group may or may not have to be activated prior to reaction with the second set of probe molecules. In a preferred embodiment, the Y functional groups do not have to be activated and can readily react with the corresponding set of probe molecules by simply contacting the substrate surface with a solution of the second set of probe molecules for a period of time sufficient to effect the reaction of the probe molecules with the corresponding crosslinkers. In another embodiment where the crosslinkers are activated in situ, the patterned surface is first contacted with a solution of the activating agent for a sufficient period of time, rinsed free of excess activating agent under conditions that do not deactivate the crosslinkers, then contacted with a solution of the probe molecules. After the immobilization of the probe molecules, the remainder of crosslinkers that did not react with probe molecules may have to be blocked. The blocking procedure may be as previously described.

After the blocking procedure, the substrate is now ready for use as a sensor. Methods for using the sensor in diffraction-based assays will be known to those skilled in the art based on pertinent patents and literature references such as in Goh, J. B.; Loo, R. W.; McAloney, R. A.; Goh, M. C. “Diffraction-Based Assay for Detecting Multiple Analytes” Anal. Bioanal. Chem (2002) 374, 54-56.

The sensor is used in a diffraction-based assay wherein the binding of probe molecules present in a fluid to the chemical cross-linkers results in a diffraction image thereby being indicative of the probe molecules being present in the fluid. When more than one pattern of chemical cross-linkers are used to detect for more than one type of probe molecule, binding of these different molecules to the different sets of chemical crosslinkers results in a diffraction image which is different from a diffraction image observed in the absence of binding of probe molecules to the cross-linkers. The diffraction image associated with each of the different cross-linker patterns arises from light hitting the pattern and the image due to one pattern will be different than the image associated with the one or more other cross-linker patterns. Similarly, molecules which bind to the probe molecules themselves may be detected in liquids as well using the same principle.

The present invention will now be illustrated using the following non-limiting examples.

### EXAMPLES

### Example 1

### Patterning of H₂N(CH₂CH₂O)₈CH₂CH₂COOH on NHS-ester Surface

Stamps made with either polyolefin plastomer (POP) or poly(dimethylsiloxane) (PDMS) with surface relief pattern were cleaned by sonication in 2:1 ethanol/deionized water for 5 minutes. The stamps were dried with a gentle stream of nitrogen and inked with a solution of H₂N(CH₂CH₂O)₈CH₂CH₂COOH (0.1 mM in 3:1 ethanol/deionized H₂O, pH adjusted to 10 with 1M NaOH) by putting enough volume of solution such that the patterned area of the stamp was totally covered. After 10 minutes, the solution was siphoned off and the stamps were dried with a gentle stream of nitrogen gas. The dried stamps were put in contact with the substrate surface functionalized with NHS-ester groups and left in contact for 5 minutes, then peeled off. The stamped substrates were exposed to a solution of Me(OCH₂CH₂)₁₁CH₂CH₂NH₂ (0.4 mM in deionized H₂O, pH adjusted to 10 with 1M NaOH) by putting a sufficient volume to cover the entire substrate surface for 30 minutes. The substrates were rinsed with deionized H₂O and sonicated in deionized H₂O for 5 minutes.

### Example 2

### Use of Substrate with Patterned H₂N(CH₂CH₂O)₈CH₂CH₂COOH in Diffraction-Based Assay

The substrate patterned with H₂N(CH₂CH₂O)₈CH₂CH₂COOH prepared as in example 1 was put in a solution of N-Ethyl-N′(3-dimethylaminopropyl)carbodiimide (EDC) and N-hydroxysuccinimide (NHS), 100 and 25 mM respectively, in deionized water for 15 hours. The substrate was then rinsed with distilled H₂O and dried with a gentle stream of nitrogen.

To make a fluid cell, a piece of glass slide was put against the patterned surface of the substrate using two pieces of double-sided sticky tape such that the two pieces of tape sandwiched between the glass slide and the substrate surface defined a channel for liquid to flow through and wet the patterned area of the substrate surface.

The fluid cell was mounted on a diffraction assay set-up. The intensity changes were monitored during the different phases of the assay. Initially the fluid cell was filled with buffer (MES, 25 mM pH 6). The buffer solution was replaced with a solution of anti-rabbit IgG (25 ug/mL in MES buffer) resulting in an increase in intensity of the diffraction signal indicating the solution-phase immobilization of the anti-rabbit IgG to the patterned H₂N(CH₂CH₂O)₈CH₂CH₂COOH. After immobilization was complete, the fluid cell was rinsed with MES buffer then blocked with a solution of bovine serum albumin (BSA) (5 mg/mL in MES). The fluid cell was again rinsed with MES buffer which was then replaced with a solution of rabbit anti-goat IgG (100 ug/mL in MES) resulting in an increase in intensity of the diffraction signal indicating the binding of the rabbit anti-goat IgG to the immobilized anti-rabbit.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

Therefore what is claimed is:

1. A sensor for immobilizing at least one type of probe molecules in patterns on a substrate, comprising:
a substrate having a surface with pre-selected areas of the surface patterned with at least one chemical crosslinker, X¹—R¹—Y¹, wherein X¹is a chemical functional group that can chemically bind with the surface, R¹ is a chemical moiety that serves as a spacer to provide distance between the surface and the probe molecules to be immobilized and also reduce non-specific interactions, and Y¹ is a chemical functional group which can form a strong interaction, either covalent or non-covalent, with the probe molecules;
remaining areas of the substrate not patterned with the at least one chemical crosslinker X¹—R¹—Y¹ being coated with blocking molecules, X²—R², wherein X² is a chemical functional group that can covalently react with the surface which may or may not be the same as X¹, and R² is a chemical moiety that reduces non-specific interactions and may or may not be the same as R¹, wherein contacting the patterned surface with the probe molecules in solution effects immobilization of the probe molecules through a strong interaction between the probe molecules and the Y¹-chemical functional group of the at least one chemical crosslinker X¹—R¹—Y¹.

2. The sensor according to claim 1 wherein said surface contains moieties rendering it an electrophilic surface, and wherein X¹ is a nucleophilic chemical functional group that can covalently react with the substrate surface.

3. The sensor according to claim 2 wherein X¹ is selected from the group consisting of amines, hydrazides, hydroxylamines and thiols.

4. The sensor according to claim 1 wherein said surface contains moieties rendering it a nucleophilic surface, and wherein X¹ is an electrophilic chemical functional group that can covalently react with the substrate surface.

5. The sensor according to claim 4 wherein X¹ is selected from the group consisting of carboxylic acids and its activated forms, epoxides, trialkoxysilanes, dialkoxysilanes, and chlorosilanes.

6. The sensor according to claim 1 wherein R¹ is a moiety that is selected to be compatible with probes which are biomolecules and minimizes non-specific interactions.

7. The sensor according to claim 1 wherein R¹ is comprised of an alkyl chain, from about 2 to about 200 atoms in length, which is optionally interrupted by heteroatoms and/or aryl groups and/or cycloalkyl groups.

8. The sensor according to claim 1 wherein functional group Y¹ is selected from the group consisting of acid chloride, mixed anhydride, N-hydroxysuccinimidyl (NHS) ester, pentafluorophenyl (PFP) ester, hydroxybenzotriazole (HObt) ester,imidazolide, epoxide, aldehyde, alpha-halo carbonyl, amine, hydrazide, and isocyanate.

9. The sensor according to claim 1 wherein said at least one chemical crosslinker is at least two chemical crosslinkers, X¹—R¹—Y¹ and X³—R³—Y³, wherein the patterns defined by the two chemical crosslinkers are different and distinct from each other, wherein X¹ and X³ are chemical functional groups that can covalently react with the surface and may or may not be the same, wherein R¹ and R³ are chemical moieties that serve as spacers to provide distance between the surface and the probe molecules to be immobilized and also helps to minimize non-specific interactions and may or may not be the same, and wherein Y¹ and Y³ are chemical functional groups that can form strong interactions, either covalent or non-covalent, with the probe molecules and may or may not be the same;
remaining areas of the substrate not patterned with said at least two crosslinkers being coated with blocking molecules, X²—R², wherein X² is a chemical functional group that can covalently react with the surface which may or may not be the same as X¹ or X³, and R² is a chemical moiety that helps minimize non-specific interactions and may or may not be the same as R¹ or R³, wherein contacting the patterned surface with a solution containing a first probe molecule effects immobilization of first probe molecules through a strong interaction between the first probe molecules and the Y¹-functional group of the chemical crosslinker X¹—R¹—Y¹, and wherein contacting the patterned surface with a solution containing a second probe molecule effects immobilization of said second probe molecule through a strong interaction between the probe molecules and the Y³-functional group of the chemical crosslinker X³—R³—Y³.

10. The sensor according to claim 9 wherein said surface contains moieties rendering it an electrophilic surface, and wherein X³ is a nucleophilic chemical functional group that can covalently react with the substrate surface.

11. The sensor according to claim 10 wherein X³ is selected from the group consisting of amines, hydrazides, hydroxylamines and thiols.

12. The sensor according to claim 9 wherein said surface contains moieties rendering it a nucleophilic surface, and wherein X³ is an electrophilic chemical functional group that can covalently react with the substrate surface.

13. The sensor according to claim 12 wherein X³ is selected from the group consisting of carboxylic acids and all its activated forms, epoxides, trialkoxysilanes, dialkoxysilanes, and chlorosilanes.

14. The sensor according to claim 9 wherein R³ is a moiety that is selected to be compatible with probes which are biomolecules and minimizes non-specific interactions.

15. The sensor according to claim 14 wherein R³ is comprised of an alkyl chain, from about 2 to about 200 atoms in length, which is optionally interrupted by heteroatoms and/or aryl groups and/or cycloalkyl groups.

16. The sensor according to claim 14 wherein functional group Y³ is selected from the group consisting of acid chloride, mixed anhydride, N-hydroxysuccinimidyl (NHS) ester, pentafluorophenyl (PFP) ester, hydroxybenzotriazole (HObt) ester,imidazolide, epoxide, aldehyde, alpha-halo carbonyl, amine, hydrazide, and isocyanate.

17. The sensor according to claim 1 for use in a diffraction-based assay wherein binding of molecules present in a fluid to probe molecules in the at least one at least one set of chemical crosslinkers results in a diffraction image which is different from a diffraction image observed in the absence of binding of molecules to the probe molecules.

18. A method for fabricating substrates with immobilized probe molecules in a pattern, comprising:
patterning pre-selected portions of a surface of a substrate with chemical crosslinkers, X¹—R¹—Y¹, wherein X¹ is a chemical functional group that can covalently react with the surface, R¹ is a chemical moiety that serves as a spacer to provide distance between the surface and the probe molecules to be immobilized and also helps to minimize non-specific interactions, and Y¹ is a chemical functional group which can form a strong chemical interaction, either covalent or non-covalent, with the probe molecules;
exposing the substrate to blocking molecules, X²—R², wherein X² is a chemical functional group that can covalently react with the surface which may or may not be the same as X¹, and R² is a chemical moiety that helps minimize non-specific interactions and may or may not be the same as R¹ so that areas of the substrate not patterned with the crosslinker X¹—R¹—Y¹ is coated with the blocking molecules X²—R²; and
contacting the patterned surface with the probe molecules in solution to effect strong chemical interaction between the Y¹ chemical functional groups of the cross linkers and the probe molecules thereby immobilizing the probe molecules attached thereto.

19. The method according to claim 18 wherein said surface contains moieties rendering it an electrophilic surface, and wherein X¹ is a nucleophilic chemical functional group that can covalently react with the substrate surface.

20. The method according to claim 19 wherein X¹ is selected from the group consisting of amines, hydrazides, hydroxylamines and thiols.

21. The method according to claim 18 wherein said surface contains moieties rendering it a nucleophilic surface, and wherein X¹ is an electrophilic chemical functional group that can covalently react with the substrate surface.

22. The method according to claim 21 wherein X¹ is selected from the group consisting of carboxylic acids and all its activated forms, epoxides, trialkoxysilanes, dialkoxysilanes, and chlorosilanes.

23. The method according to claim 18 wherein R¹ is a moiety that is selected to be compatible with probes which are biomolecules and minimizes non-specific interactions.

24. The method according to claim 23 wherein R¹ is comprised of an alkyl chain, from about 2 to about 200 atoms in length, which may or may not be interrupted by heteroatoms and/or aryl groups and/or cycloalkyl groups.

25. The method according to claim 18 wherein functional group Y¹ is selected from the group consisting of acid chloride, mixed anhydride, N-hydroxysuccinimidyl (NHS) ester, pentafluorophenyl (PFP) ester, hydroxybenzotriazole (HObt) ester,imidazolide, epoxide, aldehyde, alpha-halo carbonyl, amine, hydrazide, and isocyanate.

26. A method for fabricating a substrate with immobilized probe molecules in a pattern, comprising:
patterning pre-selected portions of a surface of the substrate with at least two types of chemical crosslinkers, X¹—R¹—Y¹ and X³—R³—Y³, wherein patterns defined by the two crosslinkers are different and distinct from each other, wherein X¹ and X³ are chemical functional groups that can covalently react with the surface and may or may not be the same, wherein R¹ and R³ are chemical moieties that serve as spacers to provide distance between the surface and the probe molecules to be immobilized and also helps to minimize non-specific interactions and may or may not be the same, and wherein Y¹ and Y³ are chemical functional groups that can form strong interactions, either covalent or non-covalent, with the probe molecules and may or may not be the same;
remaining areas of the substrate not patterned with the chemical crosslinkers X¹—R¹—Y¹ being coated with blocking molecules, X²—R², wherein X² is a chemical functional group that can covalently react with the surface which may or may not be the same as X¹ or X³, and R² is a chemical moiety that helps minimize non-specific interactions and may or may not be the same as R¹ or R³, wherein contacting the patterned surface with first probe molecules in solution effects immobilization of the first probe molecules through a strong interaction between the first probe molecules and the Y¹-functional group of the chemical crosslinkers, X¹—R¹—Y¹, and wherein contacting the patterned surface with a solution containing a second probe molecule effects immobilization of said second probe molecules through a strong interaction between the second probe molecules and the Y³-functional group of the chemical crosslinker X³—R³—Y³.

27. The method according to claim 26 wherein said surface contains moieties rendering it an electrophilic surface, and wherein X¹ is a nucleophilic chemical functional group that can covalently react with the substrate surface.

28. The method according to claim 27 wherein X¹ is selected from the group consisting of amines, hydrazides, hydroxylamines and thiols.

29. The method according to claim 26 wherein said surface contains moieties rendering it a nucleophilic surface, and wherein X¹ is an electrophilic chemical functional group that can covalently react with the substrate surface.

30. The method according to claim 29 wherein X¹ is selected from the group consisting of carboxylic acids and its activated forms, epoxides, trialkoxysilanes, dialkoxysilanes, and chlorosilanes.

31. The method according to claim 26 wherein R¹ is a moiety that is selected to be compatible with probes which are biomolecules and minimizes non-specific interactions.

32. The method according to claims 26 wherein R³ is a moiety that is selected to be compatible with probes which are biomolecules and minimizes non-specific interactions.

33. The method according to claims 26 wherein R³ is comprised of an alkyl chain, from about 2 to about 200 atoms in length, which may or may not be interrupted by heteroatoms and/or aryl groups and/or cycloalkyl groups.

34. The method according to claim 26 wherein functional group Y³ is selected from the group consisting of acid chloride, mixed anhydride, N-hydroxysuccinimidyl (NHS) ester, pentafluorophenyl (PFP) ester, hydroxybenzotriazole (HObt) ester,imidazolide, epoxide, aldehyde, alpha-halo carbonyl, amine, hydrazide, and isocyanate.

35. The method according to claim 28 for use in a diffraction-based assay wherein binding of probe molecules present in a fluid to said at least one chemical crosslinker results in a diffraction image which is different from a diffraction image observed in the absence of binding of probe molecules to said at least one chemical crosslinker.
